(19) United States
(12) Patent Application Publication
(10) Pub. No.: US 2018/0206820 A1
(43) Pub. Date: Jul. 26, 2018

(54) ULTRASOUND APPARATUS AND METHOD

(71) Applicant: CARESTREAM HEALTH, INC., Rochester, NY (US)

(72) Inventors: Ajay ANAND, Rochester, NY (US); Bhaskar S. RAMAMURTHY, Los Altos, CA (US)

(21) Appl. No.: 15/876,711

(22) Filed: Jan. 22, 2018

Related U.S. Application Data
(60) Provisional application No. 62/450,696, filed on Jan. 26, 2017.

(51) Int. Cl. A61B 8/00; A61B 8/08

(52) U.S. Cl. CPC A61B 8/4461; A61B 8/5223; A61B 8/5207; A61B 8/145; A61B 8/4272; A61B 8/54; A61B 8/463

(57) ABSTRACT

A method for ultrasound imaging generates an interleaved ultrasound beam pattern that alternates transmission between a focused ultrasound signal and a plane wave ultrasound signal during a scan. Reflected signal data from the focused ultrasound signal is directed to a first signal processing path that executes delay/sum processing and generates successive lines of image data. Reflected signal data from the plane wave ultrasound signal is directed to a second signal processing path that generates a full plane of image data. Pixel location and timing for data from the first signal processing path are synchronized with location and timing for the second signal processing path. The combined, synchronized image data from the first and second signal processing paths can be displayed, stored, or transmitted.

### CROSS REFERENCE TO RELATED APPLICATIONS

This application claims the benefit of U.S. Provisional application U.S. Ser. No. 62/450,696, provisionally filed on Jan. 26, 2017, entitled “ULTRASOUND APPARATUS AND METHOD”, in the name of Ajay Anand et al. and incorporated herein in its entirety.

### TECHNICAL FIELD

The disclosure relates generally to the field of medical diagnostic ultrasound systems and methods and more particularly to apparatus and methods that provide ultrafast imaging.

### BACKGROUND

Ultrasound imaging systems/methods are well known. See for example U.S. Pat. No. 6,705,995 (Poland) and U.S. Pat. No. 5,370,120 (Oppelt). All of the above-identified references are incorporated herein by reference in their entirety.

Conventional ultrasound imaging apparatus can have one or more transducers, transmit and receive beamformers, and various processing and display components used for generating and presenting the acquired images. A transmit beamformer supplies electrical waveform signals to the transducer arrays on the hand-held probe, which, in turn, generates the associated ultrasonic signals. Objects in the path of the transducer signals scatter ultrasound energy back to the transducers, which then generate receive electrical signals. The receive electrical signals are delayed for selected times specific to each transducer, so that ultrasonic energy scattered from selected regions adds coherently, while ultrasonic energy from other regions has no perceptible impact. Array processing techniques used for generating and processing received signals in this way are termed “beamforming” and are well known to those in the ultrasound imaging field.

Today's more advanced ultrasound systems can offer the sonographer a number of imaging options for obtaining image content that is suitable for patient assessment and diagnostics. Among options selectable by the operator or pre-programmed by the system designer are different types of beamforming algorithms. Selection of the beamforming sequence that best meets the requirements for a particular exam can be based on a number of factors, including imaging frame rate, relative noise levels, and various imaging characteristics.

Ultrasound imaging systems have typically used a conventional method of beamforming referred to as serial line-by-line imaging in the literature. In such a conventional system, the imaging is usually performed by sequential insonification of the medium using focused beams. Each focused beam allows the reconstruction of one image line or a few lines (for example, up to 16 in multi-line imaging). A typical 2D image is made of a few tens of lines (64 to 512). The frame rate of the imaging mode is set by the time required to transmit a beam, receive and process the backscattered echoes from the medium, and repeat this for all the lines of the image. A number of clinical imaging modes have been developed on this architecture, and it remains the mainstay of imaging today. In these systems, the image formation approach implemented is commonly referred to as Delay and Sum (DAS) or delay/sum beamforming.

With advances in computing power, another ultrasound beamforming architecture has emerged. This is often referred to as Ultrafast imaging. An introduction and background of this new ultrasound imaging technology is provided, for example, in the reference: “Ultrafast Imaging in Biomedical Ultrasound” in IEEE Transactions on Ultrasonics, Ferroelectrics, and Frequency Control, 61(1):102-119, January 2014, by M. Tanter and M. Fink, edited by Prof. Oleg Minin, incorporated herein in its entirety.

In Ultrafast imaging, instead of forming an image line-by-line as in conventional imaging, the entire image can be formed from a limited number of transmitted beams that use plane or divergent waves. In such a system, the image frame rate is no longer limited by the number of lines reconstructed using focused waves, but by the time of flight taken for a single plane wave pulse to propagate through the medium and return to the transducer.

With an ultrafast imaging architecture, a variety of new transmit excitation schemes are used. These include the use of plane and diverging beams that insonify a large region of the tissue. On the receive side, to achieve ultrafast imaging, systems benefit from the ability to capture and store the raw RF channel data (i.e. data from each transducer element) to reconstruct the image pixel by pixel over the entire field of view. The channel data is processed by high performance hardware such as GPUs (Graphic Processing Units) and FPGAs (Field Programmable Gate Arrays) or DSPs (Digital Signal Processors), or a combination of these, to implement novel image reconstruction algorithms, typically referred to as pixel-based beamforming. Since no transmit beamforming is applied with the plane or diverging beam transmit sequences, image and contrast resolution suffer if the image is formed with a single transmit firing. To overcome this aspect, overlapping transmit beams are used that insonify a given position from multiple directions. As a result, dynamic transmit focusing can be achieved, wherein every location in the image is in focus, compared to one or a few locations in a conventional static transmit focus imaging paradigm.

There are benefits and challenges with implementing ultrafast imaging systems on ultrasound systems, particularly if implemented in a commercial environment. Some view at least one benefit of ultrafast imaging to include substantially high frame rates without compromise on imaging resolution, and ability to implement quantitative and parametric imaging modes that are not possible without access to the raw channel data. On the other hand, transmit paradigms typically used in ultrafast imaging may not be well suited for some imaging modes. One example is harmonic imaging. First, the intensity of harmonic signals, particularly tissue harmonic signals, is very low relative to fundamental frequency signals. This is because harmonic generation is proportional to the square of the acoustic pressure at the target. Since the transmit beams (such as plane wave and diverging beams) used to best exploit the ultrafast imaging architecture are not as narrowly focused as conventional transmitted signals, the pressure of the acoustic energy field developed is far below that which is present at the focus of a standard focused transmit beam, and may even be insufficient to produce diagnostically useful harmonic images.

In clinical ultrasound systems, Harmonic imaging is typically used as part of multi-mode or duplex imaging sequences where it is combined with other mode sequences (e.g. B-mode fundamental, Color, M-mode). With known methods, the entire duplex sequence including the harmonic mode would be performed using a single beamforming approach, whereas it might be beneficial to perform duplex scanning using a combination of approaches (e.g. the non-harmonic mode using ultrafast imaging approaches while relying on DAS for harmonic imaging).

It can be appreciated that there can be significant value in providing ultrasound solutions that take advantage of multiple beamforming modes, while allowing flexibility and control for mode selection to the operator.

### SUMMARY

An object of the present disclosure is to advance the art of ultrasound imaging and overall system operation.

These objects are given only by way of illustrative example, and such objects may be exemplary of one or more embodiments of the invention. Other desirable objectives and advantages inherently achieved may occur or become apparent to those skilled in the art. The invention is defined by the appended claims.

According to one aspect of the disclosure, there is provided a method for ultrasound imaging comprising: generating an interleaved ultrasound beam pattern that alternates transmission between a focused ultrasound signal and a plane wave ultrasound signal during a scan; directing reflected signal data from the focused ultrasound signal to a first signal processing path that executes delay/sum processing and generates successive lines of image data; directing reflected signal data from the plane wave ultrasound signal to a second signal processing path that generates a full plane of image data; synchronizing pixel location and timing for data from the first signal processing path to the second signal processing path; and displaying, storing, or transmitting the combined, synchronized image data from the first and second signal processing paths.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 shows a schematic of an exemplary ultrasound system.

FIG. 3 illustrates a Sonographer using an exemplary ultrasound system.

FIG. 4 shows a displayed ultrasound image.

FIG. 5 is a logic flow diagram for a sequence that provides parallel processing paths for focused signal and plane wave/divergent wave signal processing.

FIG. 6 is a table showing exemplary combinations for imaging modes using the processing paths of FIG. 5.

FIG. 7 is a timing diagram that shows synchronization for alternation of signals in two separate modes according to an embodiment.

FIG. 8 is a schematic diagram that shows a system architecture for interleaved beamforming operation according to an embodiment of the present disclosure.

FIGS. 9A and 9B show two different display arrangements that can be used for the image data as shown on the operator interface

### DETAILED DESCRIPTION OF THE EMBODIMENTS

The following is a detailed description of the preferred embodiments, reference being made to the drawings in which the same reference numerals identify the same elements of structure in each of the several figures.

Medical ultrasound (also known as diagnostic sonography or ultrasonography) is a diagnostic imaging technique based on the application of ultrasound, used to display internal body structures such as tendons, muscles, joints, vessels and internal organs.

Reference is hereby made to US Patent Application Publication No. 2015/0141821 by Yoshikawa et al. entitled “Ultrasonic Diagnostic Apparatus and Elastic Evaluation Method” incorporated herein by reference.

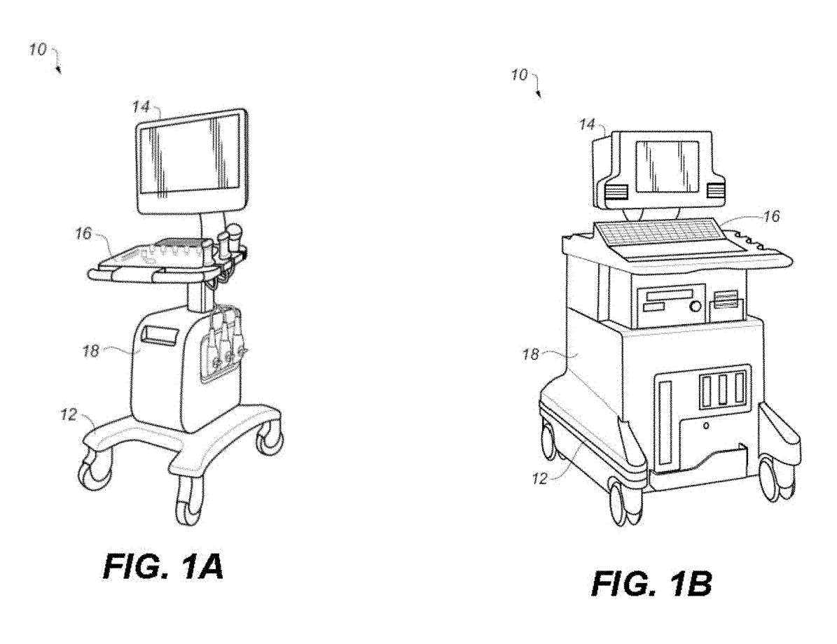

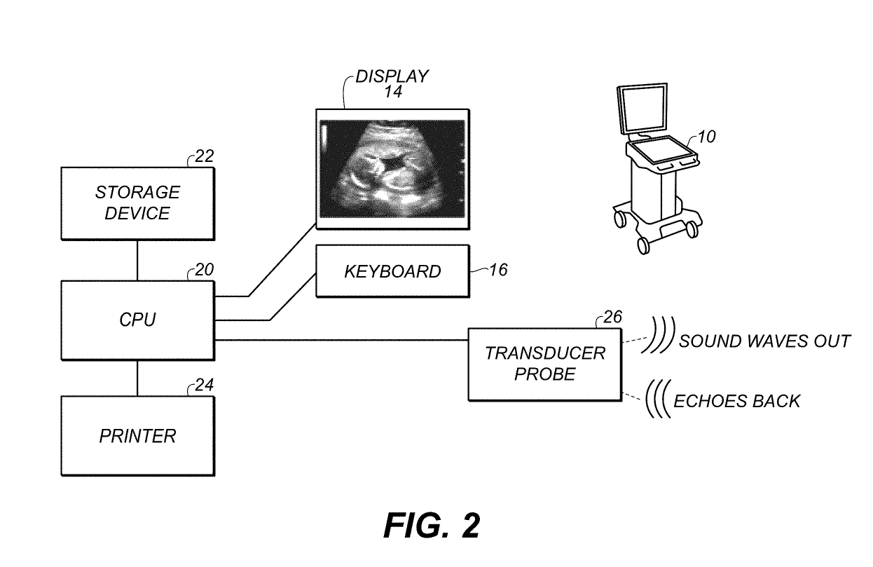

FIGS. 1A, 1B, 2, and 3 show exemplary ultrasound systems 10 including a cart/base/support 12, a display monitor 14, an input device (such as keyboard 16 or mouse), and a generator 18. The display 14 can also be a touchscreen to function as an input device for entry of commands to a controller 40 that controls ultrasound system operation. As illustrated, the ultrasound system is a mobile system having wheels. As FIG. 2 shows, the ultrasound system 10 has a central processing unit CPU 20 that provides control signals and processing capabilities. CPU 20 is in signal communication with display 14 and interface device 16, as well as with a storage device 22 and an optional printer 24. A transducer probe 26 provides the ultrasound acoustic signal and generates an electronic feedback signal indicative of tissue characteristics from the echoed sound.

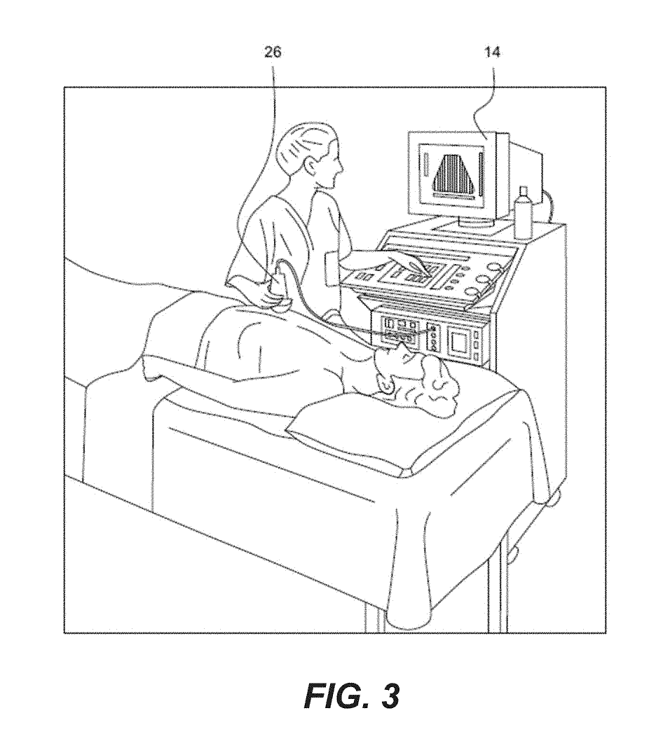

FIG. 3 shows an example of an ultrasound image displayed on the display 14.

Ultrasound is sound wave energy with frequencies higher than those audible to the human ear. Ultrasonic images, also known as sonograms, are made by directing pulses of ultrasound into tissue using a probe. The sound echoes off the tissue; with different tissues reflecting varying degrees of sound. These echoes are recorded and displayed as an image to the operator.

Different types of images can be formed using sonographic instruments. The most well-known type is a B-mode image, which displays the acoustic impedance of a two-dimensional cross-section of tissue. Other types of images can display blood flow, motion of tissue over time, the location of blood, the presence of specific materials, the stiffness of tissue, or the anatomy of a three-dimensional region.

Accordingly, the system of FIGS. 1A and 1B is configured to operate within at least two different ultrasound modes. As such, the system provides means to switch between the at least two different ultrasound modes. Such a two-mode configuration and means for switching between modes are well known within the ultrasound technology.

Clinical modes of ultrasound used in medical imaging include the following:

A-mode: A-mode (amplitude mode) is the simplest type of ultrasound. A single transducer scans a line through the body with the echoes plotted on screen as a function of depth. Therapeutic ultrasound aimed at a specific tumor or calculus also uses A-mode emission to allow for pinpoint accurate focus of the destructive wave energy.

B-mode or 2D mode: In B-mode (brightness mode) ultrasound, a linear array of transducers simultaneously scans a plane through the body that can be viewed as a two-dimensional image on screen. Sometimes referred to as 2D mode, this mode is effective for showing positional and dimensional characteristics of internal structures and is generally the starting point for exam types that use other modes.

C-mode: A C-mode image is formed in a plane normal to a B-mode image. A gate that selects data from a specific depth from an A-mode line is used; the transducer is moved in the 2D plane to sample the entire region at this fixed depth. When the transducer traverses the area in a spiral, an area of 100 cm² can be scanned in around 10 seconds.

M-mode: In M-mode (motion mode) ultrasound, pulses are emitted in quick succession. With each pulse, either an A-mode or B-mode image is acquired. Over time, M-mode imaging is analogous to recording a video in ultrasound. As the organ boundaries that produce reflections move relative to the probe, this mode can be used to determine the velocity of specific organ structures.

Doppler mode: This mode makes use of the Doppler effect in measuring and visualizing blood flow.

Color Doppler: Velocity information is presented as a color-coded overlay on top of a B-mode image. This mode is sometimes referred to as Color Flow or color mode.

Continuous Doppler: Doppler information is sampled along a line through the body, and all velocities detected at each point in time are presented (on a time line).

Pulsed wave (PW) Doppler: Doppler information is sampled from only a small sample volume (defined in 2D image), and presented on a timeline.

Duplex: a common name for the simultaneous presentation of 2D and (usually) PW Doppler information. (Using modern ultrasound machines, color Doppler is almost always also used; hence the alternative name Triplex.).

Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.

Harmonic mode: In this mode a deep penetrating fundamental frequency is emitted into the body and a harmonic overtone is detected. With this method, noise and artifacts due to reverberation and aberration are greatly reduced. Some also believe that penetration depth can be gained with improved lateral resolution; however, this is not well documented.

A Sonographer, ultrasonographer, clinician, practitioner, or other clinical user, is a healthcare professional (often a radiographer but may be any healthcare professional with the appropriate training) who specializes in the use of ultrasonic imaging devices to produce diagnostic images, scans, videos, or 3D volumes of anatomy and diagnostic data.

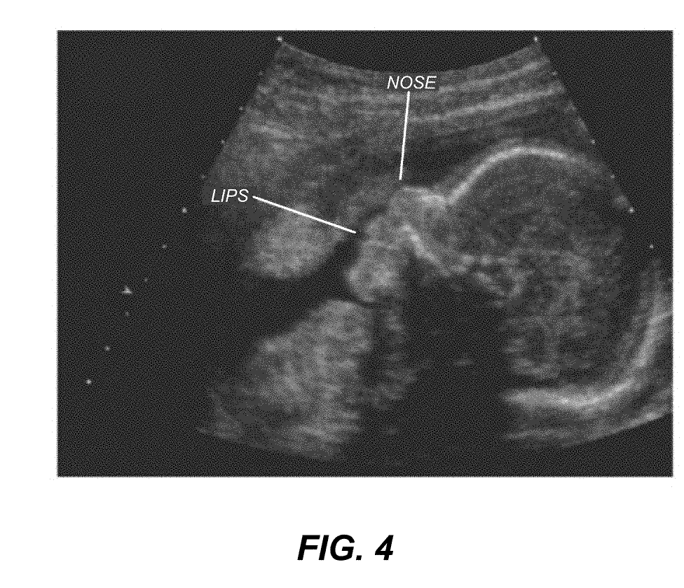

FIG. 4 shows a displayed ultrasound image in grey scale. Such an image would be captured using a grey scale mode, for example, using B-mode.

Harmonic imaging relies on imaging the harmonic signal components that are generated as the incident acoustic wave propagates through the tissue due to the non-linear properties of tissue. The higher the acoustic pressure in the tissue, the more pronounced the harmonic signal content. Since conventional beamforming types typically rely on narrowly focused transmit signals, they are inherently better suited for harmonic imaging, when compared to plane wave and diverging beam approaches. However, there can be a number of instances in which plane wave and diverging beam beamforming signal types can be advantaged. To help improve system operability, embodiments of the present disclosure address the problem of beamforming selection by beginning a scan using a first beamforming type, analyzing the data generated following the first beamforming paradigm as this data is generated, and dynamically determining whether or not to switch to another beamforming type. A novel aspect of the present disclosure is a proposed method wherein the optimal beamforming type for the specific clinical environment (considering factors such as anatomy, body habitus, and ultrasound system capability) is automatically selected, or is suggested to the operator, in order to provide a useful clinical outcome.

Diverging beam and plane wave beamforming allow high temporal resolution color flow data to be acquired. The same data, saved over a short time period, can be used to create a Spectral Doppler waveform. Advantageously, the acquired data is available over the entire image rather than at only a single location.

It is understood that the different ultrasound beamforming types and associated imaging algorithms have advantages as well as limitations. For example, diverging beams and plane wave beamforming signals used for ultrafast ultrasound processing provide high frame rates, but do not, in conventional implementations, provide focus for the transmitted signal, which can result in poorer spatial and contrast resolution. High frame rates, such as equivalent to several hundred frames per second (FPS) are achievable with diverging and plane wave beamforming signal sequences, because these sequences allow reconstruction over larger regions with relatively fewer number of signal emissions, such as a single transmission, or a set of plane wave transmissions, each at a different angle with relation to the imaged tissue. In the context of the present disclosure, the phrase “diverging beam/plane wave” is used to represent emission using either divergent or plane beamforming types, since there is often no formal distinction needed between the types. This beamforming type is distinguished from conventional, serial delay/sum beamforming and processing.

Recent developments in beam transmission and signal processing have provided methods to overcome some of the shortcomings of plane wave and divergent beam imaging. Coherent plane wave compounding has been introduced, for example, wherein plane waves are transmitted into tissue from multiple overlapping directions and are coherently summed to improve image resolution by reducing side lobes. Conversely, serial delay-sum (or delay/sum) beamforming or more conventional beamforming schemes allow tightly focused beams, and therefore provide better spatial resolution for a targeted region of interest (ROI), but at lower frame rates. Some modes such as harmonic imaging, while feasible, are more challenging with diverging and plane wave beams.

Some conventional ultrasound hardware has been adapted for use in ultrafast imaging applications. However, the conventional architecture of earlier systems makes it difficult to take advantage of the speed and accuracy that is available with ultrafast imaging techniques. Moreover, conventional systems adapted in this way do not offer the capability to interleave ultrafast processing with conventional serial delay/sum ultrasound processing, constraining the operator to choose either one or the other. In contrast, embodiments of the present disclosure allow the operator to combine conventional and ultrafast imaging without compromising either imaging capability.

The present disclosure describes an architecture and framework whereby interleaved signal modes, such as those noted above, can be provided by the beamforming paradigm, providing performance (such as using serial imaging or ultra-fast imaging) best suited for each case. By interleaving different types of ultrasound beams, an embodiment of the present disclosure enables the ultrasound system control logic to acquire image content in multiple modes and to synchronize the storage, transmission, and display of image data obtained using different types of beamforming signals.

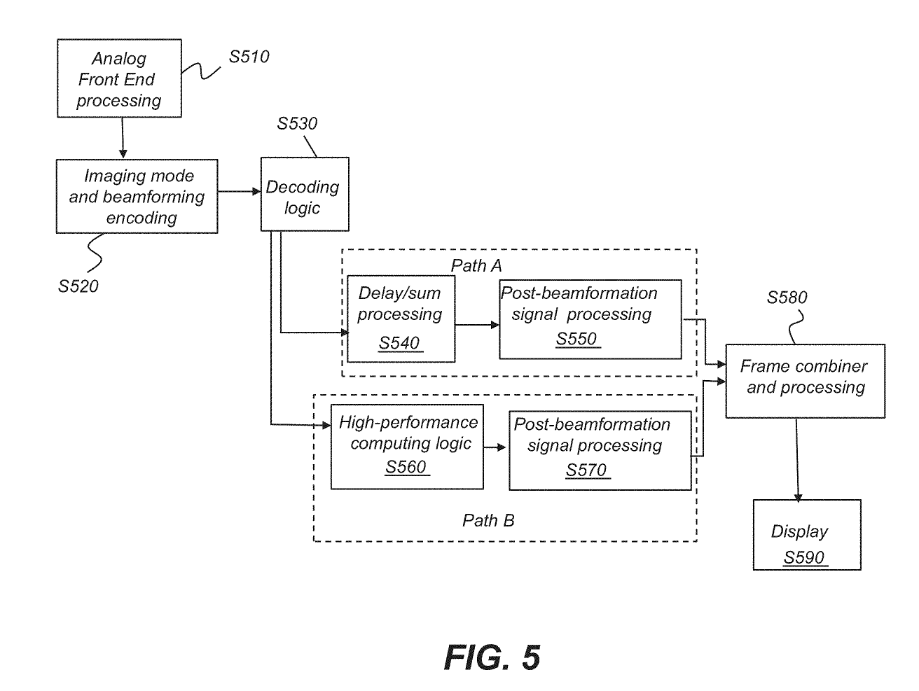

A particular aspect of the disclosure relates to how the data for the individual sub-modes are gathered, processed, stored and displayed or rendered on the clinical display. Different beamforming schemes work best for the various clinical image modes described previously. To address the need for multiple mode imaging with suitable system performance and simplified workflow, embodiments of the present disclosure provide a hybrid architecture for interleaved signal imaging, as shown in the flow diagram of FIG. 5. As can be appreciated from this diagram, the receive path is modified and forms a distinguishable feature of the disclosure.

As illustrated in FIG. 5, there are 2 parallel receive data paths (Path A and B) to process, in parallel, the raw channel data received from the transducer elements. Path A refers to the serial imaging signal path for focused beam data, while Path B is used for ultrafast imaging using plane wave or divergent wave data. Depending on the required imaging mode, the operation of each of the signal paths can be adapted or customized.

Applicants describe an example for Duplex imaging that provides Harmonic B-mode imaging along with Color mode imaging, wherein harmonic B-mode and Color mode data are generated and rendered on a frame-interleaved or line-interleaved basis. When Duplex imaging with Harmonic B-mode is initiated, the transmit sequence is set up with the appropriate pulses required for Harmonic B-mode and Color mode. In an exemplary sequence, Harmonic B-mode is effected using focused beams with the transmit focus placed at a narrow region within the tissue. Color mode imaging is typically best performed using ultrafast imaging techniques, with plane signal transmission. The harmonic B-mode and Color mode transmit signals are interleaved in a pre-determined manner, with the signal scheduler firing the appropriate signal pulses accordingly. For receive signal processing shown in FIG. 5, harmonic B-mode imaging uses Path A. In this way, the harmonic signal path uses serial imaging that is well suited for the narrow focused transmit beams. Color flow uses Path B, with plane or divergent beam transmission.

Referring to the individual detailed steps in the signal delivery, acquisition, and processing sequence of FIG. 5, an analog front end processing step S510 generates the transponder signal output and acquires the image data for each processing mode and beamforming type. Analog front end processing can include TGC (transponder gain control), anti-aliasing software, and digital-to-analog conversion (DAC) processes. The front-end can be configured to interleave delay/sum imaging signals with plane-wave or divergent beam imaging, with the two different signal types sent and received by the transponder, but directed along different imaging paths, as described subsequently.

An imaging mode and beamforming type selection step S520 selects the imaging mode type and beamforming type for the analog front end and specifies the corresponding encoding according to operator selection, or predetermined setup, of conventional or ultrafast processing. A decoding logic step S530 determines the appropriate processing path for the acquired interleaved signals obtained from the transponder, directing conventional delay/sum ultrasound signal content to Path A, and ultrafast ultrasound processing for the plane or divergent wave signal content to Path B, as described herein.

Path A processing has a delay/sum processing step S540 that performs conventional delay and sum beamforming, such as using FPGA or conventional software configuration for conventional ultrasound image extraction. A subsequent post-beamformation processing step S550 then executes the appropriate post-processing for the conventional delay/sum signal.

Path B in the FIG. 5 sequence processes plane wave or divergent wave imaging data used in ultrafast ultrasound imaging. A high-performance computing logic step S560 employs GPU or high-capacity FPGA hardware to perform the needed computation for plane- or divergent-beam imaging. A subsequent post-beamformation processing step S570 then executes the appropriate post-processing for the plane-wave or divergent beam imaging signal. A full plane of image data at a time can be generated using ultrafast ultrasound techniques.

Following Path A and Path B processing, a frame combiner step S580 assembles image content from both the conventional signal and ultrafast ultrasound signal processing. A display step S590 then displays results of image processing for either or both processing paths.

For the processing sequence shown in FIG. 5, each received analog image signal from an individual transponder element passes through the same analog data path, undergoing A/D conversion at step S510. The converted digital ultrasound data then initiates the image mode and beamforming encoding, providing data described for step S530. As part of step S530, depending on the type of transmit signal that was used, the data can be tagged with the correct mode type (e.g. Color mode, b-mode, harmonic etc) and flagged to identify the beamforming path it takes. These choices can be pre-determined on the system using configuration files/parameters set up during system configuration, or can be user selectable from the keyboard or via other human computer interface devices. This tagged data is fed to decoding logic step S530 where, depending on the beamforming data path requested, the data is streamed onto the corresponding path A or B. For processing speed and flexibility, the decode logic is preferably implemented in FPGA or other hardware.

It is possible in certain embodiments that the decode logic and the delay/sum beamforming can be implemented on the same physical piece of hardware. This can depend on the complexity and capacity of the apparatus hardware and programmed logic.

Following beamforming, the data is processed using the post-beamforming signal and image processing functions described in FIG. 5. The data is then fed to frame combiner step S580 where the resulting image from each of the beamforming paths is aggregated in the right order before presentation to the display in display step S590. Display step S590 can show combined or fused image content from multiple imaging modes and from both paths A and B.

It is noted that although the acquisition is interleaved, the architecture presented does not require synchronization between the two paths A and B on a per data sample basis (typically 25 ns at 40 MHz). Instead, computing logic must be incorporated in the frame combiner circuitry, so that frame combiner step S580 waits until all of the data corresponding to a frame has arrived (from each of the processing paths) before presenting it to the display. Frame combiner step S580 provides both spatial and timing synchronization, as described subsequently.

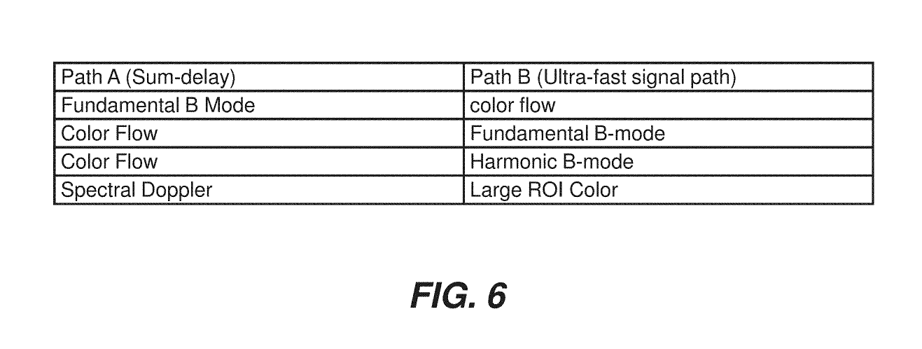

FIG. 6 provides a list of possible combined modes according to the path utilized for each mode.

Pixel Synchronization (Spatial Registration of Information from Paths A and B)

Pixel synchronization allows the two types of image content from delay/sum imaging using focused ultrasound beams and ultrafast imaging using plane wave or divergent wave signals to be readily processed, synchronized, and displayed. In the final display, the images formed by the two signal processing paths A and B in FIG. 5 should be synchronized at a pixel level. In other words, each pixel of the final display must carry information generated by the two signal processing paths where the information is generally about the same region in the tissue. The controller ensures that two pieces of information (one from each signal processing path) are spatially synchronized. An example of how this can be performed is presented below:

(a) The spatial coordinates of the image pixels that contain the fused information from the 2 paths, typically as a 800 by 600 or equivalent 2D grid, are first defined as a look up table. This can be referred to as the global image coordinate system.

(b) For Path A of FIG. 5, a reference coordinate system and origin is defined. The choice of this coordinate system and origin can depend on the type of transducer and the scanning geometry. The acoustic data is received and processed in this path-native coordinate system until the final stage of scan conversion and presentation to the display. In the scan conversion stage, the spatial data from this native coordinate system is mapped onto the global image coordinate system defined in (a) above.

(c) Similarly for Path B, the acoustic signal processing steps can produce spatial data in the global coordinate system defined in Step (a). With the ultra-fast imaging paradigm, the scan conversion step is not explicitly needed but rather the output of the acoustic signal processing steps would directly be a 2D spatial matrix.

(d) Once the data streams from (b) and (c) have been mapped to (a), decision logic can be applied, similar to the “priority” control in conventional ultrasound systems, that determines whether the data from Path A or B is presented on a given pixel. The decision can be made, for example, based on the energy content of the specific pixel, statistical variance, or other statistical parameters.

### Timing Synchronization

Just as the information from the two signal paths are synchronized spatially, it is also desirable to synchronize the posting of the information from each signal path. It is possible that, in some cases, the timing of the transmission is impacted due to the inability to completely flush the data from the signal processing path. An example transmit sequence is presented below. The example is based on a focused transmit-based B-mode imaging via Path A and color-flow imaging via Path B:

(a) The focused transmit beams for B-mode imaging via Path A are fired sequentially one after another. In response to each transmit signal, the receive data is routed through Path A, and the scan-converted data is stored in an image buffer.

(b) Interleaved with the focused transmission and acquisition, the transmit sequence to create an ultrafast imaging frame for Path B is fired. The emitted signal can be a plane or divergent wave signal. For a Color flow sequence, this could also entail a rapid series of transmissions at the same spatial location that forms the ensemble. The data for this excitation is routed via Path B, and processed to create a Color flow frame and stored in the image buffer as in (a).

(c) Once the corresponding image frame outputs from (a) and (b) are obtained, the data can be displayed on the screen. In case latency causes delay in the output from (b) reaching the image buffer in time, a decision can be made by a logic component to either discard that frame output and begin the next transmit sequence, or else wait for output from (b) to arrive before beginning the next transmit. Consequently, the image would be updated only after the corresponding frames from both paths are available to maintain timing synchronization.

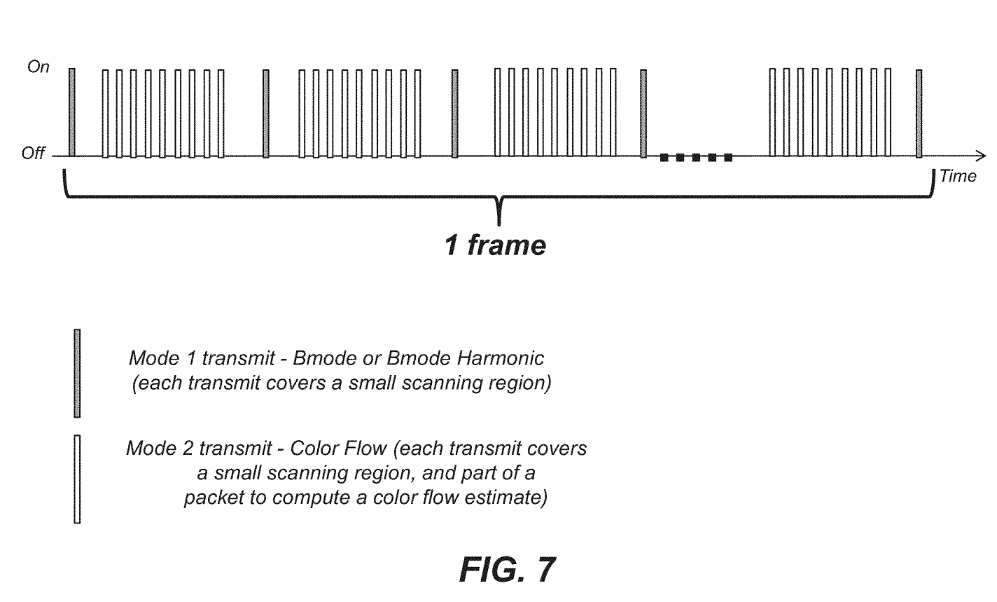

The graph of FIG. 7 shows a timing diagram for interleaved signal generation and processing to generate ultrasound data content for one image frame from multiple modes using a single transducer. Acoustic signals for a first mode, Mode 1, are repeatedly alternated with acoustic signals for acquiring ultrasound data in a second mode, Mode 2. Each Mode 1 signal covers a small scanning region, such as using a focused ultrasound beam. Each Mode 2 signal is shown as a series of pulsed signals that combine to provide color flow data in the arrangement shown.

### Designating a Beamforming Mode

According to an embodiment of the present disclosure, the controller, control logic processor, or computer that controls ultrasound imaging operation provides a predefined, default set of combined modes and also allows the operator to override default settings and specify beamforming types to be used for any particular combination of modes. A table similar to that shown in FIG. 6 is displayed on a user interface terminal, with accompanying controls, pull-down menus, and other selection utilities for mode combination and beamforming type setting for each mode.

In addition to allowing operator selection, the control logic processor may also measure image quality and, based on data analysis, determine whether or not imaging with plane wave or divergent wave beamforming achieves acceptable results.

Data analysis can be performed to extract an image metric with information such as contrast resolution, spatial resolution, penetration, frame rate, and the like. A determination can be made as to whether or not the extracted metric is suitable, such as within an acceptable range of values, for the given anatomy and body part. If the metric is acceptable, scanning can continue using the same diverging beam/plane wave beamforming signal type. If one or more metrics are not suitable, it can be beneficial to switch to the delay-sum (focused beam) beamforming signal type or to another beamforming variant suitable for harmonic imaging or other imaging mode. Suitability can be determined by comparing the values of the metric(s) against one or more stored threshold values, for example.

Optionally, instead of switching the beamforming type, the system could automatically adjust filter settings for the current beamforming type, based on image analysis. It should be noted that automated switching of beamforming type change can be optional; as an alternative, a message or prompt is provided to the operator allowing entry, confirmation, or override of the proposed change in beamforming type.

### System Architecture for Interleaved Ultrasound Operation

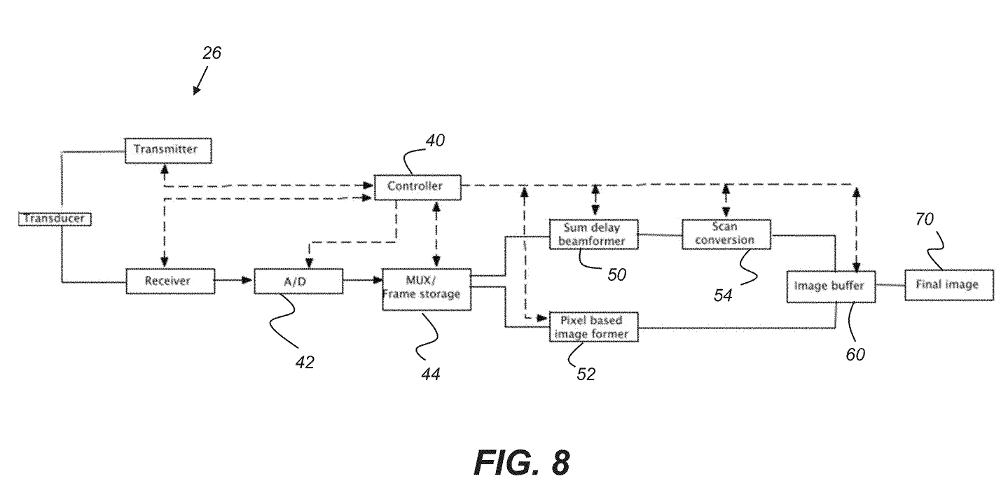

At least one mode for implementing the beam interleaving signal delivery and processing of the present disclosure includes a high performance medical ultrasound system where the architecture is designed to support both focused beam and plane wave/divergent wave signal paths concurrently. FIG. 8 shows an exemplary system architecture for an ultrasound apparatus that is configured to execute, in interleaved form, a sequence using both focused delay/sum imaging and harmonic imaging, and plane wave or divergent wave imaging.

In the FIG. 8 schematic, transducer probe 26 logic is configured with transmitter and receiver circuitry for both focused and plane wave and/or divergent wave modes. A controller 40, such as a microprocessor or other control logic processor executes the logic that controls transducer probe 26 signal emission and reception and controls A/D (analog-to-digital) circuitry 42 that converts the received echo signal and provides it to a multiplexer (MUX) 44 that coordinates image frame storage. Controller 40 also provides signals that control a sum/delay beamformer 50 and pixel-based image former 52. A scan conversion circuit 54 provides conversion of the incoming scan data and provides this data to an image buffer 60 for storage and display as the final image 70.

### User Interface

Several techniques can be used to make the decision about which imaging path to utilize. In one technique, the optimal choices are preprogrammed within the ultrasound system. As an example, a table may be created within the stored programming of the ultrasound system, containing the rules defining the paths to utilize. Thus continuing the example described previously, a rule may state that B-mode fundamental imaging may always use divergent or plane waves. Another rule may state that harmonic imaging may always use focused modes. An operator would not necessarily need to know what transmit paradigms or imaging path is being used.

On the other hand, the decision on which path to use may be selected by the user through a user interface. Thus, for example, the user may choose between divergent waves, plane waves or conventional focused modes for fundamental B-mode images. Alternatively, a preference from the user or the clinical application being imaged might, in turn, determine which imaging mode is to be invoked. For example, a certain range of frame rates might use serial imaging via FIG. 5 Path A while another range might invoke ultrafast imaging through Path B.

This disclosure describes a methodology/architecture to interleave multiple modes of imaging in a frame.

This disclosure proposes a framework wherein Color and Harmonic imaging are both implemented using the optimal ultrasound imaging path.

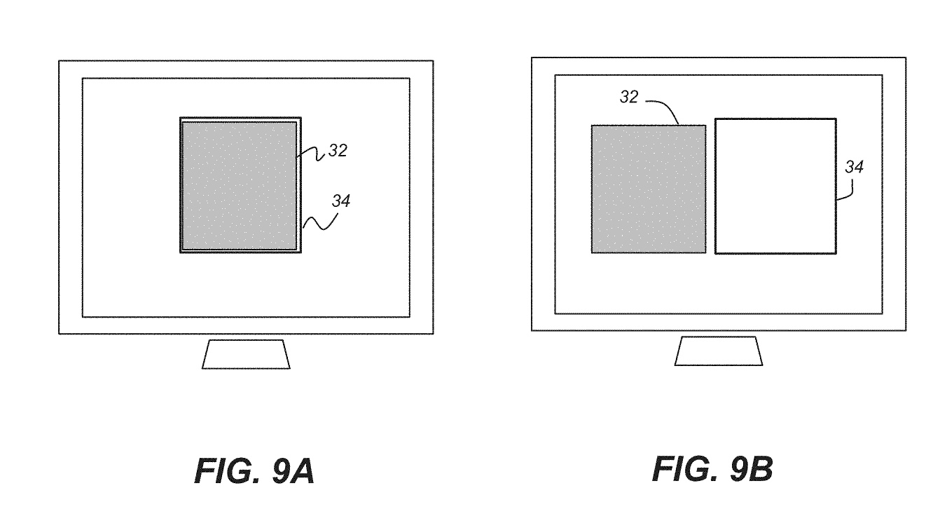

The schematic diagrams of FIGS. 9A and 9B show two different display arrangements that can be used for the image data as shown on the operator interface. FIG. 9A shows an overlaid arrangement, wherein an image 32 generated in one mode is combined and overlaid on an image 34 generated in a different mode. This type of display arrangement may be most suitable for some imaging mode combinations, such as for displaying combined Color mode and B-mode imaging. FIG. 9B shows a side-by-side arrangement, in which pixel content from different modes is not combined, but shown for ready reference or comparison. The side-by-side arrangement may be most suitable for showing results from an imaging exam wherein Doppler and B-mode images are concurrently obtained.

According to an embodiment of the present disclosure, an operator command on the user interface allows the viewer to select the image from either mode individually, such as for full screen display. Tabs or other on-screen utilities then enable the viewer to select which image displays at a particular time.

Embodiments of the present disclosure can be applied in hand-carried systems where traditionally it has been considered expensive to incorporate high performance hardware, or alternatively has limited power consumption and computing capabilities to do extensive ultrafast imaging. In such architectures, it may be desirable to have embedded hardware/firmware that supports serial imaging to do part of the processing using serial imaging which is potentially less expensive and to execute other functions using ultrafast imaging.

The present invention can be a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method for ultrasound imaging comprising:
generating an interleaved ultrasound beam pattern that alternates transmission between a focused ultrasound signal and a plane wave ultrasound signal during a scan;
directing reflected signal data from the focused ultrasound signal to a first signal processing path that executes delay/sum processing and generates successive lines of image data;
directing reflected signal data from the plane wave ultrasound signal to a second signal processing path that generates a full plane of image data at a time;
synchronizing pixel location and timing for data from the first signal processing path to data in the second signal processing path; and
displaying, storing, or transmitting the combined, synchronized image data from the first and second signal processing paths.

2. The method of claim 1 further comprising providing color flow image content using the full plane of image data.

3. The method of claim 1 further comprising obtaining the lines of image data from B-mode imaging.

4. The method of claim 1 further comprising obtaining the lines of image data from harmonic imaging.

5. The method of claim 1 further comprising detecting image quality along the second signal processing path and switching data to the first signal processing path according to the quality detection.

6. The method of claim 1 wherein displaying the combined image data comprises displaying a single image.

7. The method of claim 1 wherein displaying the combined image data comprises displaying a plurality of images simultaneously.

8. An apparatus for ultrasound imaging comprising:
a) a signal transducer that emits a repeated sequence that interleaves a focused ultrasound beam with a plane wave or divergent wave beam;
b) a control logic processor that controls the transducer and that directs a received signal from the transducer to either a first signal processing path for delay/sum processing or a second signal processing path for processing of a full image plane;
c) an image buffer in signal communication with the control logic processor and configured to combine image content from the first and second signal processing paths; and
d) a display that is configured to display the image content from the image buffer.

9. The apparatus of claim 8 wherein the control logic processor further comprises software that determines whether to direct the received signal to the first or second signal processing path.

10. A method for ultrasound imaging comprising:
generating an interleaved scanning beam transmission pattern that alternates between transmitting a focused beam ultrasound signal and a plane wave or diverging wave ultrasound signal;
directing reflected signal data from the focused ultrasound signal to a first signal processing path that executes delay/sum processing and generates successive lines of image data;
directing reflected signal data from the plane wave ultrasound signal to a second signal processing path that generates a full plane of image data at a time;
evaluating the image quality of the reflected signal data from the second signal processing path and changing the scanning beam transmission pattern to continuously generate a focused beam ultrasound signal according to the evaluation; and
displaying, storing, or transmitting the image data from the reflected signal data.

11. The method of claim 10 wherein changing the scanning beam transmission pattern further comprises directing the reflected signal data from the second signal processing path to the first signal processing path.

12. The method of claim 10 further comprising providing color flow image content using the full plane of image data.

13. The method of claim 10 further comprising obtaining the lines of image data from B-mode imaging.

14. The method of claim 10 further comprising obtaining the lines of image data from harmonic imaging.
